(19) United States
(12) Patent Application Publication
(10) Pub. No.: US 2023/0001013 A1
(43) Pub. Date: Jan. 5, 2023

(54) STABLE MONOMERIC INSULIN FORMULATIONS ENABLED BY SUPRAMOLECULAR PEGYLATION OF INSULIN ANALOGUES

(71) Applicant: THE BOARD OF TRUSTEES OF THE LELAND STANFORD JUNIOR UNIVERSITY, Palo Allto, CA (US)

(72) Inventors: Eric A. APPEL, Palo Alto, CA (US); Caitlin MAIKAWA, Boston, MA (US); Anton Allen Abbotsford SMITH, Kgs. Lyngby (DK)

(21) Appl. No.: 17/784,907

(22) PCT Filed: Dec. 14, 2020
(86) PCT No.: PCT/US2020/064912

§ 371 Date: Jun. 13, 2022

Related U.S. Application Data
(60) Provisional application No. 62/948,159, filed on Dec. 13, 2019.

(51) Int. Cl. A61K 47/69; A61K 38/28; A61K 47/60; A61K 47/26; A61K 47/10; A61K 47/02; A61K 38/22; A61P 3/10

(52) U.S. Cl. CPC A61K 47/6949; A61K 38/28; A61K 47/60; A61K 47/26; A61K 47/10; A61K 47/02; A61K 38/22; A61P 3/10

(57) ABSTRACT

Stable monomeric insulin formulations are enabled by supramolecular PEGylation of insulin or insulin analogues, and provide a method for treating diabetes, or managing or reducing blood glucose.

### RELATED APPLICATIONS

This application claims the benefit of the priority of U.S. Provisional Application No. 62/948,159, filed Dec. 13, 2019, which is incorporated herein by reference in its entirety.

### GOVERNMENT RIGHTS

This invention was made with government support under Grant No. R01DK119254 awarded by the National Institutes of Health. The government has certain rights in the invention.

### TECHNICAL FIELD

Provided herein are stable monomeric insulin formulations enabled by supramolecular PEGylation of insulin or insulin analogues, and method of use thereof in treating diabetes, or managing or reducing blood glucose levels.

### BACKGROUND

Over 40 million patients live with Type 1 diabetes worldwide. The loss of insulin production in these patients results in an inability to process glucose without exogenous insulin delivery through daily subcutaneous injections, or by infusion with a pump into the subcutaneous tissue. Maintaining tight glycemic control is critical to prevent severe long-term side effects, such as kidney disease, heart disease, vision loss, and limb loss. Patients must deliver a carefully calculated bolus of insulin at mealtimes to reduce blood glucose excursions; however, insulin pharmacokinetics following subcutaneous administration are poorly matched to physiologic post-prandial requirements. Even current commercial “rapid-acting” insulin analogue formulations Humalog (insulin lispro), Novolog (insulin aspart) and Apidra (insulin glulisine) exhibit delayed onset of action of ˜20-30 min, peak action at ˜60-90 min and a total duration of action of ˜3-4 hours.

Current “fast-acting” insulin analogues inhibit dimer formation and shift the equilibrium towards the monomeric state through amino acid modifications. However, the insulin monomer is unstable and rapidly aggregates to form amyloid fibrils. Insulin is primarily formulated as hexamers to prevent insulin aggregation. Novolog (aspart) and Humalog (lispro) are formulated in sodium phosphate buffer with a three-fold molar excess of zinc ion, relative to the insulin hexamer. Formulation with zinc stabilizes the hexameric state in the T₆ formation, and dissociation of the hexamer is known to be the rate-limiting step for subcutaneous absorption and onset of action. In contrast, Apidra (glulisine) is a zinc-free formulation and is formulated with the surfactant polysorbate 20 as a stabilizing agent. Apidra demonstrates slightly faster onset of action, but overall similar control of glucose levels in vivo to Novolog and Humalog, indicating that the removal of zinc alone is not enough to achieve an ultra-fast acting monomeric insulin formulation.

Excipients, the inactive ingredients in drug formulations, perform a number of functions and can facilitate improved protein stability, solubility and absorption. Formulations for insulin analogues contain multiple excipients including tonicity agents, preservatives, and stabilizing agents, which are selected to enhance insulin stability. Glycerol or sodium chloride are commonly added to insulin formulations as tonicity agents, whereas phenol and/or meta-cresol are added as parenteral preservatives. The inclusion of glycerol, a tonicity agent used in both Humalog and Novolog formulations, has been shown to increase insulin stability in formulation. Moreover, in addition to anti-microbial properties, phenol and meta-cresol stabilize the R₆ insulin hexamer by forming hydrogen bonds between dimers. This suggests that even in the absence of zinc, the phenolic preservatives may contribute to higher order insulin structures that may slow absorption from the subcutaneous space.

Formulating monomeric insulin requires new excipients which do not promote the R₆ hexamer, but still imbue insulin with sufficient stability to prevent aggregation and denaturation over time. Covalent PEGylation has been successful as a strategy to stabilize insulin in formulation, however, the extended pharmacokinetics in vivo associated with PEGylation is not desirable for rapid acting insulins. Recent research has demonstrated non-covalent modification of proteins as a strategy to enhance their stability in formulation. In particular, conjugation of a polyethylene glycol (PEG) chain to a cucubit[7]uril (CB[7]) creates a tool for non-covalent PEGylation using host-guest binding with the excipient CB[7]-PEG. CB[7]-PEG has strong binding affinities for terminal aromatic amino acids such the N-terminal phenylalanine found on insulin making it an ideal candidate for host-guest binding. The dynamic binding of CB[7]-PEG to insulin is promising as a strategy for stabilizing insulin without promoting the insulin hexamer.

Understanding the excipient choices in current commercial formulations is a critical first step in designing the next generation of ultra-fast acting insulin formulations that have potential to more closely mimic endogenous pharmacokinetics to address the above-described unmet need.

### BRIEF SUMMARY

The present invention is directed a stable monomeric insulin formulation that is achieved through selection of formulation excipients that promote the monomer state. In embodiments of the inventive approach, CB[7]-PEG can be used to stabilize these formulations so that the insulin/CB[7]-PEG complex has a faster diffusion rate than the insulin hexamer.

Current “fast-acting” insulin analogues contain amino acid modifications meant to inhibit dimer formation and shift the equilibrium of association states towards the monomeric state. However, the insulin monomer is highly unstable and current formulation techniques require insulin to primarily exist as hexamers to prevent aggregation into inactive and immunogenic amyloids. Insulin formulation excipients have thus been traditionally selected to promote insulin aggregation into the hexameric form to enhance formulation stability. The inventive approach exploits a novel excipient for the supramolecular PEGylation of insulin analogues, including aspart and lispro, to enhance the stability and maximize the prevalence of insulin monomers in formulation. Using multiple techniques employing a formulation excipients (tonicity agents and parenteral preservatives) enables insulin analogue formulations with 70-80% monomer and supramolecular PEGylation imbued stability under stressed aging for over 100 h without altering insulin association state. Comparatively, commercial “fast-acting” formulations contain less than 1% monomer and remain stable for only 10 h under the same stressed aging conditions. The inventive formulation approach enables next-generation ultra-fast insulin formulations with short duration of action that can effectively reduce the risk of post-prandial hypoglycemia in the treatment of diabetes.

### BRIEF DESCRIPTION OF THE DRAWINGS

FIG. 1A is a schematic illustrating the contributions of insulin association state on absorption kinetics; FIGS. 1B and 1C are activity curves comparing commercial “fast-acting” insulin formulations and formulations according to embodiments of the present invention.

FIGS. 2A-2C illustrate association states of lispro with different formulation excipients, where FIG. 2A provides SEC-MALS elution profiles; FIG. 2B plots number-averaged molecular weight of the distribution of insulin lispro association states; and FIG. 2C diagrammatically illustrates the ratio of monomers, dimers and hexamers in each formulation.

FIGS. 3A-3C illustrate association states of aspart with different formulation excipients, where FIG. 3A provides SEC-MALS elution profiles; FIG. 3B plots number-averaged molecular weight of the distribution of insulin lispro association states; and FIG. 3C diagrammatically illustrates the ratio of monomers, dimers and hexamers in each formulation.

FIGS. 4A and 4B are plots of SEC-MALS normalized by cumulative weight for different formulations zinc-free insulin lispro and aspart, respectively.

FIG. 5 is a plot of assay results for in vitro bioactivity plotted as a ratio of [pAKT]/[AKT] for each sample (n=3 cellular replicates) and an EC₅₀ regression (log(agonist) vs. response (three parameters).

FIG. 6 plots the results of acridine orange competitive binding assay of insulin aspart with CB[7]-PEG indicating binding with the CB[7] moiety.

FIGS. 7A-7E are plots comparing in vitro stability of insulin lispro under different formulation conditions with a molar ratio of CB[7]-PEG:Lispro of 0:1, 3:1, and 5:1 against a commercial Humalog control, where FIG. 7A shows lispro in phosphate buffer with saline (0.9%); FIG. 7B, lispro in phosphate buffer with glycerol (2.6%); FIG. 7C, lispro in phosphate buffer with glycerol (2.6%) and phenol (0.25%); FIG. 7D, lispro in phosphate buffer with glycerol (2.6%) and meta-cresol (0.315%); and FIG. 7E, lispro in phosphate buffer with glycerol (2.6%) and phenoxyethanol (0.85%). FIG. 7F provides a comparison of stability by aggregation times (t_{A}).

FIGS. 8A-8E are plots comparing in vitro stability of insulin aspart under different formulation conditions with molar ratios of CB[7]-PEG:Aspart of 0:1, 3:1, and 5:1 against a commercial Novolog control, where FIG. 8A shows aspart in phosphate buffer with saline (0.9%); FIG. 8B, aspart in phosphate buffer with glycerol (2.6%); FIG. 8C, aspart in phosphate buffer with glycerol (2.6%) and phenol (0.25%); FIG. 8D, aspart in phosphate buffer with glycerol (2.6%) and meta-cresol (0.315%), FIG. 8E, aspart in phosphate buffer with glycerol (2.6%) and phenoxyethanol (0.85%). FIG. 8F provides a comparison of stability by aggregation times (t_{A}).

FIG. 9 is a comparison of blood glucose measured after injection of commercial Novolog and Zn-free aspart formulated with CB[7]-PEG (5 eq.) in fasted diabetic rats.

FIGS. 10A-10D show the DOSY-measured diffusion characteristics of commercial Humalog and Novolog in the presence of zinc ion (FIG. 10A), LGPhE and AGPhE in the presence of CB[7]-PEG (0.6 eq) (FIG. 10B). FIGS. 10C and 10D respectively show the increased diffusion for insulin LGPhE and AGPhE formulated with 0.6 eq. CB[7]-PEG compared to commercial formulation conditions.

FIG. 11 shows the diffusion characteristics of Zn-free aspart under commercial formulation conditions as measured using DOSY.

FIGS. 12A and 12B plot the weight-average molecular weights of zinc-free insulin lispro and aspart, respectively, measured by SEC-MALS.

FIG. 13 shows ¹H 2D DOSY spectra demonstrating insulin/CB[7]-PEG binding for lispro (left) and aspart (right). Groups include: (i) insulin, (ii) insulin and free PEG₅ₖ, (iii) CB[7]-PEG and (iv) insulin/CB[7]-PEG complex.

FIG. 14 shows ¹H 2D DOSY spectra demonstrating insulin/CB[7]-PEG binding.

### DETAILED DESCRIPTION OF EMBODIMENTS OF THE INVENTION

Definitions: In describing the present invention, the following terms will be employed, and are intended to be defined as indicated below.

As used in this specification and the appended claims, the singular forms “a”, “an” and “the” include plural referents unless the content clearly dictates otherwise. Thus, for example, reference to “a protein” includes a mixture of two or more such proteins, and the like.

The term “about,” particularly in reference to a given quantity, is meant to encompass deviations of plus or minus five percent.

The terms “variant,” “analog” and “mutein” refer to biologically active derivatives of the reference molecule that retain desired activity, such as insulin or amylin activity for use in the treatment of type 1 or type 2 diabetes as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same insulin or amylin biological activity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, praline, phenylalanine, methionine, tryptophan; and (4) uncharged polar—glycine, asparagme, glutamine, cysteine, senne threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and KyteDoolittle plots, well known in the art.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

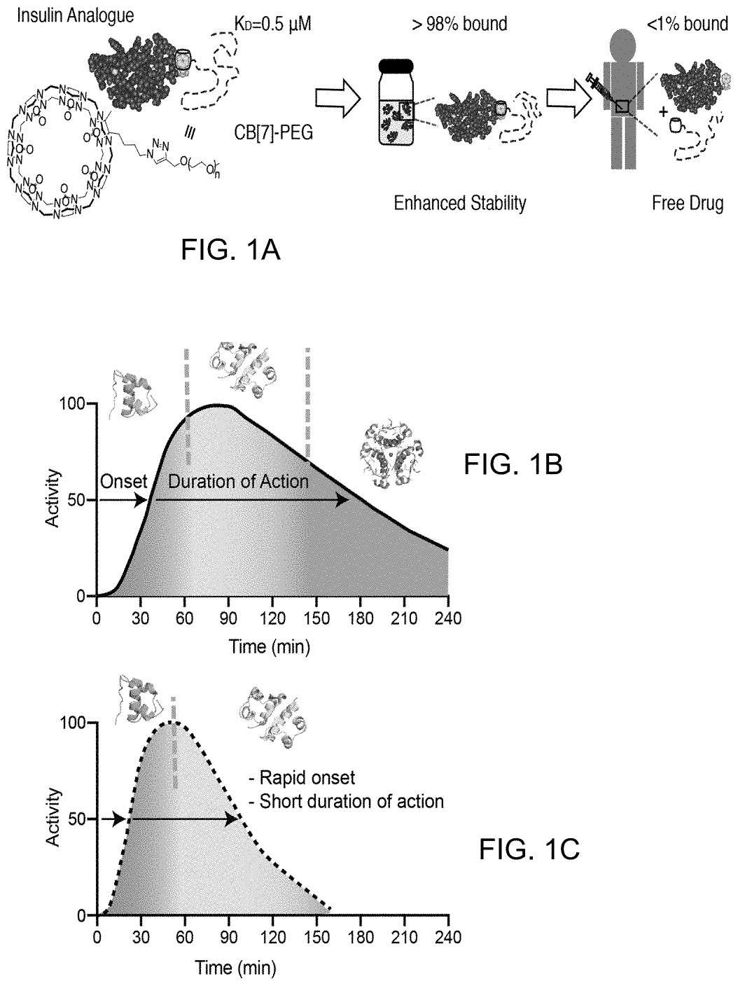

Referring to FIGS. 1A and 1B, existing insulin formulations contain a mixture of hexamers, dimers and monomers, which, upon subcutaneous injection, dissociate and are absorbed at different rates, shown diagrammatically in FIG. 1A. Non-covalent PEGylation is enabled by the strong specific binding between CB[7]-PEG and the N-terminal phenylalanine on insulin (KD=0.5 μM). Under high concentrations in formulation insulin will be greater than 98% bound, but after dilution upon subcutaneous injection less than 1% of CB[7]-PEG will remain associated with insulin. This differential absorption results in the rapid onset but long duration of action of these formulations, as indicated in the activity curve in FIG. 1B. By comparison, a completely monomeric insulin formulation would enable faster onset, and reduced duration of action, which is the next step in creating an ultra-fast acting insulin formulation, as shown in FIG. 1C. The formulations described herein are directed to this objective.

### FORMULATIONS AND METHODS OF USE

In one embodiment, a pharmaceutical composition comprises insulin or an insulin analogue; an optionally substituted cucurbit[7]uril (CB[7])—polyethylene glycol (PEG) conjugate; a tonicity agent; and a preservative; wherein no more than 20% of the insulin or insulin analogue exists as hexamers in the pharmaceutical composition.

In another embodiment, a pharmaceutical composition comprises insulin or an insulin analogue; an optionally substituted cucurbit[7]uril (CB[7])—polyethylene glycol (PEG) conjugate; a tonicity agent; and a preservative, wherein the preservative is phenoxyethanol.

In some embodiments, the insulin analog is insulin aspart. In other embodiments, the insulin analog is insulin lispro. In still other embodiments, the insulin analog is insulin glulisine.

The tonicity agent may be glycerol, sodium chloride, or mannitol, or a mixture thereof.

In one embodiment, the CB7-PEG conjugates are described in PCT application publication No. WO 2017/062622 by Webber et al., the entirety of which is incorporated herein by reference.

In one embodiment, CB7 is conjugated to PEG via a linker, which may be of Formula (L-1):

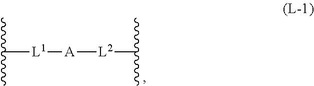

where each of L¹ and L² is independently a bond, optionally substituted alkylene, or optionally substituted heteroalkylene; and A is a bond, optionally substituted heterocyclyl, or optionally substituted heteroaryl. In some embodiments, A is optionally substituted heteroaryl. In other embodiments, A is an optionally substituted triazole moiety.

In one embodiment, the PEG has a molecular weight<1 kDa. In one embodiment, the PEG has a molecular weight between 1-10 kDa, inclusive. In one embodiment, the PEG has a molecular weight of approximately 5 kDa. In one embodiment, the PEG has a molecular weight between 5-10 kDa, inclusive. In one embodiment, the PEG has a molecular weight of approximately 10 kDa. In one embodiment, the PEG has a molecular weight between 10-30 kDa, inclusive. In one embodiment, the PEG has a molecular weight of approximately 30 kDa. In one embodiment, the PEG has a molecular weight>30 kDa. In one embodiment, the PEG has a molecular weight under 100 kDa.

In one embodiment, the pharmaceutical composition further comprises a phosphate buffer. In some embodiments, the phosphate buffer is a sodium phosphate buffer.

In one embodiment, the pharmaceutical composition is substantially free of a stabilizing agent that promotes the formation or stability of insulin hexamer.

In one embodiment, the pharmaceutical composition does not comprise a stabilizing agent at an amount effective for promoting the formation or stability of insulin hexamer.

In one embodiment, the pharmaceutical composition is substantially free of zinc. In one embodiment, the pharmaceutical composition comprises no more than trace amount of zinc. In one embodiment, the pharmaceutical composition comprises no more than 0.0002 wt. % of zinc.

In one embodiment, the pharmaceutical composition is substantially free of polysorbate. In one embodiment, the pharmaceutical composition comprises no more than trace amount of polysorbate. In one embodiment, wherein the pharmaceutical composition comprises no more than 0.0002 wt. % of polysorbate.

In one embodiment, the pharmaceutical composition comprises the insulin or insulin analogue at a concentration of from about 50 U/mL to about 200 U/mL. In one embodiment, the pharmaceutical composition comprises the insulin or insulin analogue at a concentration of about 100 U/mL.

In one embodiment, the pharmaceutical composition comprises the tonicity agent at an amount of from about 0.5% to about 5% of the total weight of the pharmaceutical composition. In one embodiment, the pharmaceutical composition comprises the tonicity agent at an amount of about 2.6% of the total weight of the pharmaceutical composition. In one embodiment, the tonicity agent is glycerol.

In one embodiment, the pharmaceutical composition comprises the preservative at an amount of from about 0.2% to about 1.5% of the total weight of the pharmaceutical composition. In one embodiment, the pharmaceutical composition comprises the preservative at an amount of about 0.85% of the total weight of the pharmaceutical composition. In one embodiment, the preservative is phenoxyethanol.

In one embodiment, the molar ratio of CB[7]—PEG to the insulin or insulin analogue is from about 1:1 to about 10:1. In one embodiment, the molar ratio of CB[7]— PEG to the insulin or insulin analogue is from about 3:1 to about 5:1. In one embodiment, the molar ratio of CB[7]—PEG to the insulin or insulin analogue is at least about 3:1. In one embodiment, the molar ratio of CB[7]—PEG to the insulin or insulin analogue is at least about 5:1.

In one embodiment, at least 50% of the insulin or insulin analogue exists as monomers in the pharmaceutical composition. In one embodiment, at least 60% of the insulin or insulin analogue exists as monomers in the pharmaceutical composition.

In one embodiment, no more than 20% of the insulin or insulin analogue exists as hexamers in the pharmaceutical composition. In one embodiment, no more than 10% of the insulin or insulin analogue exists as hexamers in the pharmaceutical composition.

In one embodiment, the pharmaceutical composition further comprises amylin or an amylin analogue. In one embodiment, the amylin or an amylin analogue is pramlintide.

In one embodiment, the pharmaceutical composition enables a reduction in delay between injection and resulting decrease in blood glucose levels, as compared to an equivalent dose of human recombinant insulin or insulin analogues.

In one embodiment, the pharmaceutical composition retains a minimum of 80% activity after stressed aging at 37° C. for a minimum of 24 hours.

In one embodiment, the pharmaceutical composition is suitable for subcutaneous administration.

In one embodiment, provided herein is a method of treating diabetes, or managing or reducing blood glucose level in a subject in need thereof, comprising administering a therapeutically effective amount of a pharmaceutical composition provided herein to the subject. In some embodiments, the subject is human. The diabetes may be either type 1 diabetes or type 2 diabetes.

In one embodiment, provided herein is a kit, pump, or pen comprising a pharmaceutical composition provided herein and instruction for treating diabetes, or managing or reducing blood glucose level. In one embodiment, the kit, pump, or pen further comprises means for delivering the pharmaceutical composition to a subject.

### EXAMPLES

### Example 1: Insulin Association State by SEC-MALS

Excipient choice is important, because as in the case of Apidra (glulisine), the absence of zinc is not sufficient to result in an ultra-fast acting insulin formulation. To engineer an insulin formulation that promotes the monomer state, the effects of excipients on insulin association state under zinc-free conditions must be understood. Characterization by size exclusion chromatography with multiple angle light scattering (SEC-MALS) allows us to determine insulin molecular weight in formulation and estimate the fraction of hexamers, dimers and monomers under formulation conditions. In these studies, insulin analogues lispro and aspart were formulated with ethylenediaminetetraacetic acid (EDTA) to remove formulation zinc. EDTA forms strong complexes with zinc (K_{D}˜10×10⁻¹⁸ M) and addition of one molar equivalent of EDTA relative to the zinc ion in insulin formulations rapidly sequester the zinc, preventing it from interacting with the insulin and thus disrupting the insulin hexamer in solution. Zinc free insulin in pure water is completely monomeric (Mₙ=5.7 kDa). However, it becomes a mixture of monomers, dimers and hexamers with the addition of buffering salts. To select for a mostly monomeric insulin formulation, we evaluated the effect of tonicity agents and preservatives on insulin association state.

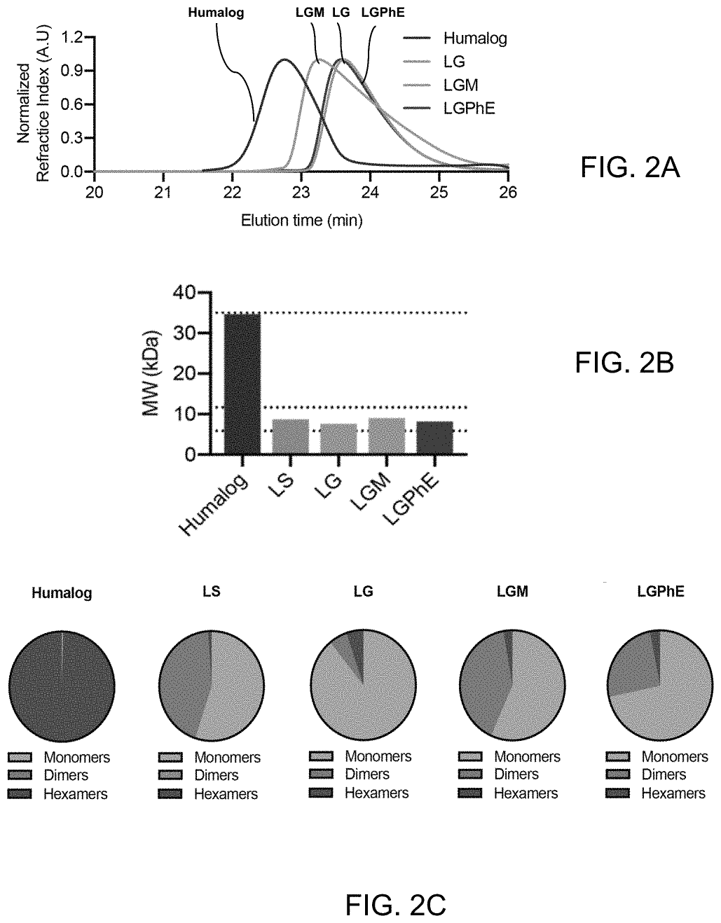

FIGS. 2A-2C illustrate the zinc-free insulin lispro association states when formulated in (i) phosphate buffer, sodium chloride (0.9%) (LS), (ii) phosphate buffer with glycerol (2.6%) (LG), (iii) phosphate buffer with glycerol (2.6%) and meta-cresol (0.315%) (LGM), and (iv) phosphate buffer with glycerol (2.6%) and phenoxyethanol (0.85%) (LGPhE). Formulations were compared against a formulation of commercial Humalog. FIG. 2A plots SEC-MALS elution profiles while FIG. 2B shows the number-averaged molecular weight of the distribution of insulin lispro association states. FIG. 2C graphically illustrates the ratio of monomers, dimers and hexamers in each formulation. Formulation details for lispro and aspart formulations are provided in Table 1

TABLE 1
Lispro formulations

| | | Buffer | | | |
|---|---|---|---|---|---|
| | Insulin | Conditions | Tonicity | Preservative | Zinc |
| | (U/mL) | (mM) | (wt. %) | (wt. %) | (wt. %) |
| Humalog | 100 U/mL | 13 mM dibasic | 1.6% glycerol | 0.315% | 0.00196% |
| (commercial) | lispro | sodium | | meta-cresol | zinc |
| | | phosphate | | | |
| Lispro + | 100 U/mL | 10 mM sodium | 0.9% sodium | — | — |
| Saline (LS) | lispro | phosphate buffer | chloride | | |
| Lispro + | 100 U/mL | 10 mM sodium | 2.6% glycerol | — | — |
| Glycerol (LG) | lispro | phosphate buffer | | | |
| Lispro + | 100 U/mL | 10 mM sodium | 2.6% glycerol | 0.315% | — |
| Glycerol + | lispro | phosphate buffer | | meta-cresol | |
| Meta-cresol | | | | | |
| (LGM) | | | | | |
| Lispro + | 100 U/mL | 10 mM sodium | 2.6% glycerol | 0.25% phenol | — |
| Glycerol + | lispro | phosphate buffer | | | |
| Phenol (LGP) | | | | | |
| Lispro + | 100 U/mL | 10 mM sodium | 2.6% glycerol | 0.85% | — |
| Glycerol + | lispro | phosphate buffer | | phenoxyethanol | |
| Phenoxyethanol | | | | | |
| (LGPhE) | | | | | |

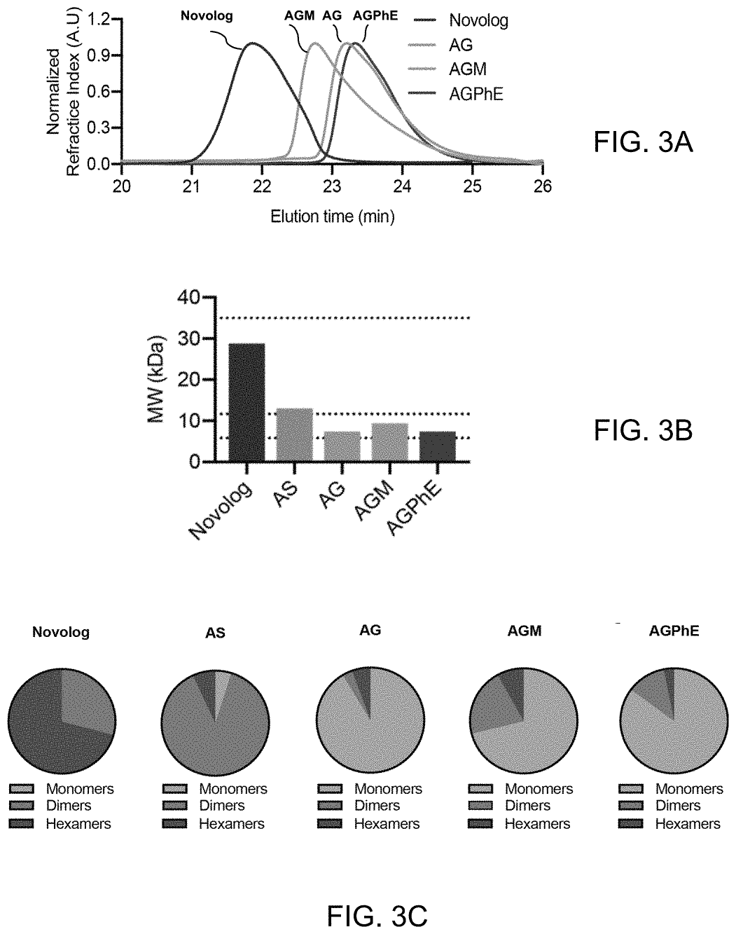

FIGS. 3A-3C illustrate zinc-free insulin aspart association states when formulated in (i) phosphate buffer, sodium chloride (0.9%) (AS), (ii) phosphate buffer with glycerol (2.6%) (AG), (iii) phosphate buffer with glycerol (2.6%) and meta-cresol (0.315%) (AGM), and (iv) phosphate buffer with glycerol (2.6%) and phenoxyethanol (0.85%) (AGPhE). Formulations were compared against a formulation of commercial Novolog. FIG. 2A plots SEC-MALS elution profiles while FIG. 2B shows the number-averaged molecular weight of the distribution of insulin aspart association states. FIG. 3C graphically illustrates the ratio of monomers, dimers and hexamers in each formulation. Formulation details for aspart formulations are provided in Table 2.

TABLE 2
Aspart formulations

| | | Buffer | | | |
|---|---|---|---|---|---|
| | Insulin | Conditions | Tonicity | Preservative | Zinc |
| | (U/mL) | (mM) | (wt. %) | (wt. %) | (wt. %) |
| Novolog | 100 U/mL | 7 mM dibasic | 1.6% glycerol | 0.172% | 0.00196% |
| (commercial) | aspart | sodium | 0.058% | meta-cresol | zinc |
| | | phosphate | sodium | 0.15% phenol | |
| | | | chloride | | |
| Aspart + | 100 U/mL | 10 mM sodium | 0.9% sodium | — | — |
| Saline (AS) | aspart | phosphate buffer | chloride | | |
| Aspart + | 100 U/mL | 10 mM sodium | 2.6% glycerol | — | — |
| Glycerol (AG) | aspart | phosphate buffer | | | |
| Aspart + | 100 U/mL | 10 mM sodium | 2.6% glycerol | 0.315% | — |
| Glycerol + | aspart | phosphate buffer | | meta-cresol | |
| Meta-cresol | | | | | |
| (AGM) | | | | | |
| Aspart + | 100 U/mL | 10 mM sodium | 2.6% glycerol | 0.25% phenol | — |
| Glycerol + | aspart | phosphate buffer | | | |
| Phenol (AGP) | | | | | |
| Aspart + | 100 U/mL | 10 mM sodium | 2.6% glycerol | 0.85% | — |
| Glycerol + | aspart | phosphate buffer | | phenoxyethanol | |
| Phenoxyethanol | | | | | |
| (AGPhE) | | | | | |

As hypothesized, all zinc-free formulations had decreased hexamer composition compared to either commercial Humalog (Mₙ=34.7 kDa; 99.6% hexamer) or commercial Novolog (Mₙ=28.8 kDa; 70.7% hexamer). Zinc-free formulations were primarily a mixture of monomers and dimers with small amounts of hexamer.

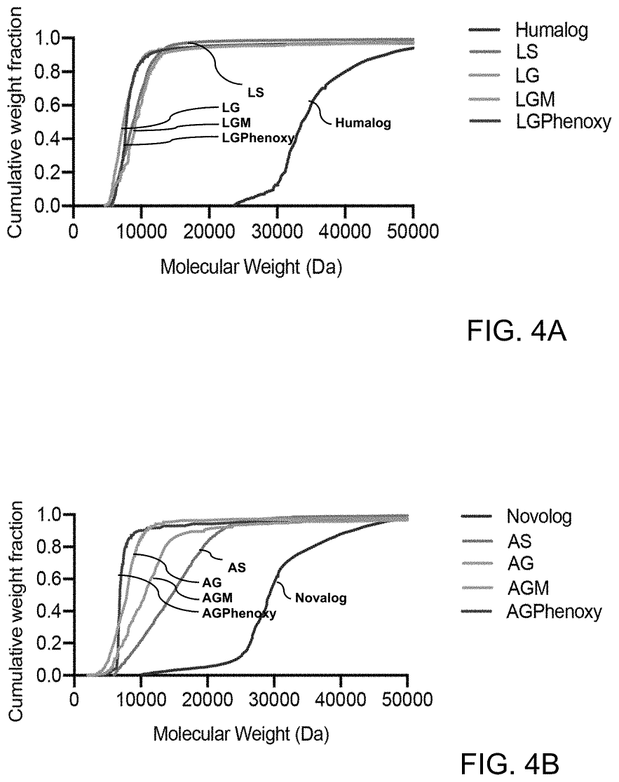

FIGS. 4A and 4B are plots of SEC-MALS normalized by cumulative weight for different formulations zinc-free insulin lispro and aspart, respectively. FIG. 4A shows zZinc-free insulin lispro association states when formulated in (i) phosphate buffer with saline (0.9%) (LS), (ii) phosphate buffer with glycerol (2.6%) (LG), (iii) phosphate buffer with glycerol (2.6%) and meta-cresol (0.315%) (LGM), and (iv) phosphate buffer with glycerol (2.6%) and phenoxyethanol (0.85%) (LGPhE). Formulations were compared against a formulation of commercial Humalog. FIG. 4B plots zinc-free insulin aspart association states when formulated in (i) phosphate buffer with saline (0.9%) (LS), (ii) phosphate buffer with glycerol (2.6%) (LG), (iii) phosphate buffer with glycerol (2.6%) and meta-cresol (0.315%) (LGM), and (iv) phosphate buffer with glycerol (2.6%) and phenoxyethanol (0.85%) (LGPhE). Formulations were compared against a formulation of commercial Novolog.

Glycerol is the most commonly used tonicity agent in insulin formulations followed closely by sodium chloride. Zinc-free lispro or zinc-free aspart were formulated in phosphate buffer (pH=7.4) with either sodium chloride (0.9%) or glycerol (2.6%). Glycerol demonstrated a lower number average molecular weight and higher percentage of monomers for both lispro (MW=7.6 kDa; 89.2% monomer) and aspart (MW=7.4 kDa; 91.4% monomer) compared to sodium chloride formulations for lispro (MW=8.7 kDa; 55.0% monomer) and aspart (MW=13 kDa; 4.8% monomer). Therefore, glycerol was maintained as the tonicity agent when evaluating the effect of preservatives on insulin association state.

While glycerol alone showed the highest proportion of monomers in formulation, insulin requires an antimicrobial preservative, because it is a multi-dose formulation. There are only several commercially used parenteral preservatives, the most common of which are benzyl alcohol, chloro-butanol, meta-cresol, phenol, methyl-paraben, propyl paraben, phenoxyethanol and thimerosal. Of these anti-microbial agents, only phenol and meta-cresol are currently used in commercial insulin formulations. However, phenolic preservatives are recognized for their promotion of R₆ insulin hexamer formation through hydrogen bonding between the hydroxyl group on phenol and the insulin dimeric pocket. To create a monomeric insulin formulation it is imperative to reduce phenol promoted R₆ hexamer formation, thus it is necessary to identify a different parenteral preservative for insulin formulations. Other research has indicated that ethanol may play a role in disrupting the insulin dimer, thus promoting the insulin monomer. This led to the selection of phenoxyethanol as an alternative anti-microbial agent. We hypothesized that the ethanol chain off the phenolic ring will create steric hindrance that disrupts the intermolecular forces leading to R₆ hexamer formation and that the ethanol side chain could further promote monomer formation through the disruption of the insulin dimer. Indeed, after glycerol alone, phenoxyethanol demonstrated the lowest number average molecular weight and highest ratio of insulin monomers in both lispro (MW=8.2 kDa; 71.5% monomer) and aspart formulations (MW=7.4 kDa; 85.2% monomer) (see FIGS. 2B, 2C, 3B, 3C). In contrast, formulation of meta-cresol had increased number average molecular weight compared to phenoxyethanol for lispro (MW=9.0 kDa; 56.7% monomer) and aspart (MW=9.4 kDa; 71.0% monomer). Work by Gast et al. has shown that phenol shows a stronger affinity for hexamer formation than meta-cresol. Therefore, it is expected that phenoxyethanol would also have a higher monomer content than a phenol based formulation. While the proportion of hexamers between formulations with phenoxyethanol and meta-cresol were similar, meta-cresol formulations had a higher percentage of hexamers and dimers combined. Since the R₆ hexamer is a dynamic structure held together by hydrogen bonds, insulin that originates as a hexamer may dissociate and appear in the dimer form in the SEC-MALS.

### Example 2: Stability of Monomeric Insulin with CB[7]-PEG

As insulin monomer content increases, formulation stability becomes more challenging and alternative stabilizing excipients are needed to increase insulin stability in the absence of zinc. The current commercial zinc-free insulin analogue, Apidra, contains both meta-cresol and the surfactant polysorbate 20 which aid formulation stability, but meta-cresol promotes the R₆ hexamer. Non-covalent PEGylation has potential as a stabilizing excipient that will not promote hexamer formation and will leave insulin monomers unmodified to act in vivo. Previous studies have shown that non-covalent PEGylation with cucurbit[7]uril-poly(ethylene glycol) (CB[7]-PEG) imbues long-term insulin stability without reducing insulin activity in vivo. Lispro formulated with glycerol/phenoxyethanol (LGPhE) and 3 molar equivalents CB[7]-PEG with respect to insulin demonstrated equal in vitro bioactivity compared to commercial Humalog in a dose response assay of AKT phosphorylation (Humalog EC₅₀=1×10⁻⁴ mg/mL; LGPhE EC₅₀=6×10⁻⁵ mg/mL).

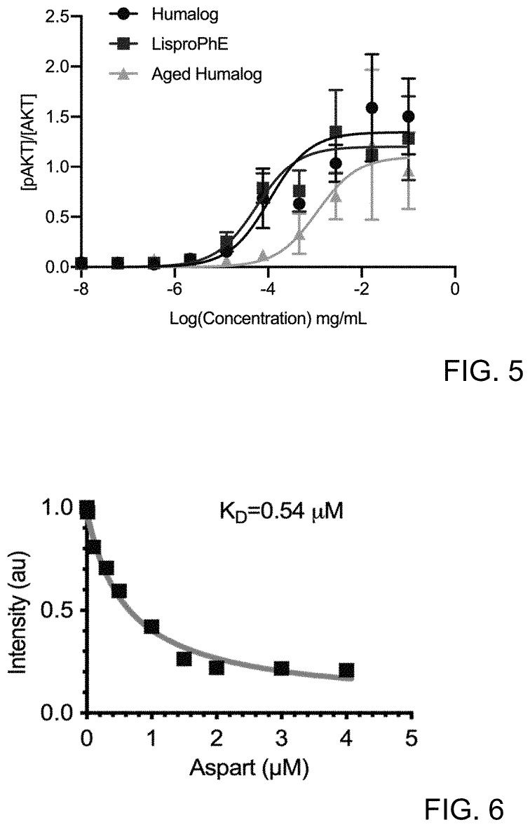

In vitro activity was evaluated by assaying for phosphorylation of Ser⁴⁷³ on AKT for (i) Humalog, (ii) zinc-free insulin lispro formulated with glycerol/phenoxyethanol and a molar excess of CB[7]-PEG compared to insulin (LGPhE), (iii) aged Humalog (4 days shaking at 37° C.). LPhE3 demonstrated unaltered activity from commercial Humalog (Humalog EC₅₀=1×10′ mg/mL; LGPhE EC₅₀=6×10⁻⁵ mg/mL; p=0.3) and both demonstrated increased activity compared to the aged control (Aged EC₅₀=1×10⁻³ mg/mL; Humalog/Aged p=0.01; LGPhE/Aged p=0.0004). Data shown are mean±s.d for n=3 experimental replicates. FIG. 5 illustrates the results, plotted as a ratio of [pAKT]/[AKT] for each sample (n=3 cellular replicates) and an EC₅₀ regression (log(agonist) vs. response (three parameters)) was plotted using GraphPad Prism 8.

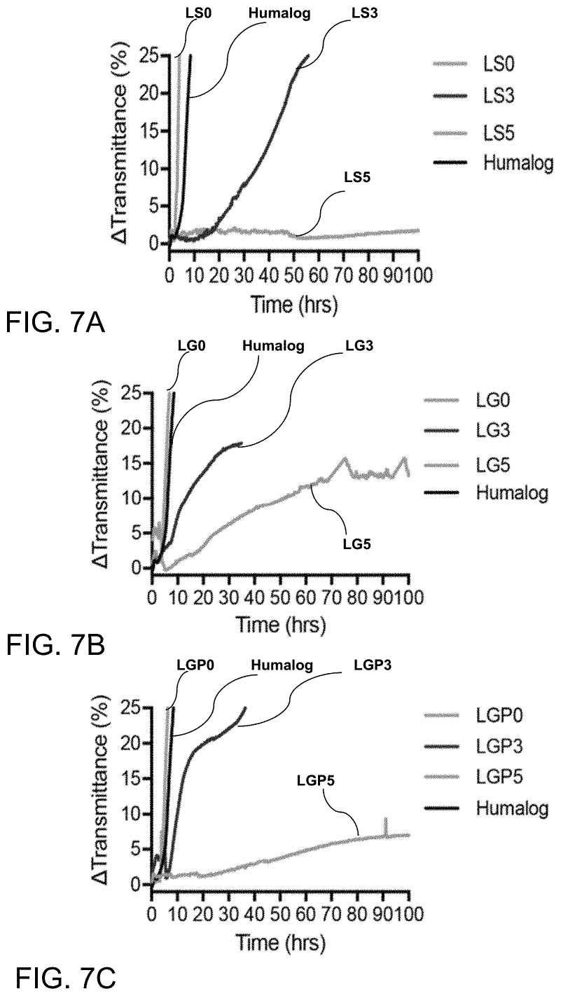

CB[7] has a binding affinity of 0.54 μM to insulin aspart (FIG. 6), such that under typical formulation concentrations, the CB[7]-PEG/insulin complex will be greater than 98% bound, but immediately upon dilution following subcutaneous administration, less than 1% of the CB[7]-PEG will remain associated with insulin.

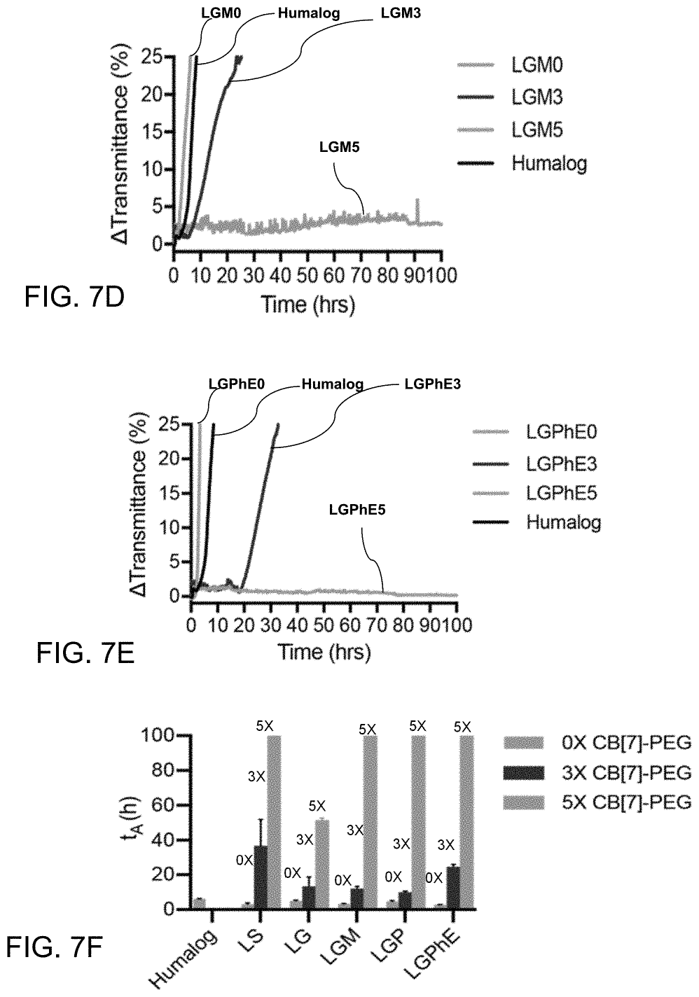

To demonstrate the utility of non-covalent PEGylation with CB[7]-PEG as a strategy to stabilize monomeric insulin long-term under a variety of formulation conditions, zinc-free insulin lispro and aspart were formulated in phosphate buffer with either (i) saline (LS), (ii) glycerol (LG), (iii) glycerol/phenol (LGP), (iv) glycerol/meta-cresol (LGM), or (v) glycerol/phenoxyethanol (LGPhE). The different combinations of excipients were evaluated to determine which provided the greatest stability. The results are plotted in FIGS. 7A-7E, respectively. CB[7]-PEG was added to formulations in excess to insulin at either 3 molar equivalents or 5 molar equivalents. Glycerol was chosen as a tonicity agent for combination with preservatives (including phenol, meta-cresol, phenoxyethanol) due to the increased affinity for the insulin monomer in the presence of glycerol compared to sodium chloride (FIGS. 7B-7E). Zinc-free lispro under all formulation conditions was less stable than the commercial Humalog formulation, thus demonstrating the need for additional stabilizing agents. A three-fold excess of CB[7]-PEG to insulin resulted in increased stability, which ranged from 1.5-fold increase in the LGP3 formulation (FIG. 7B) to a 6-fold increase in the LS3 formulation (FIG. 7A). Addition of five-fold excess of CB[7]-PEG to lispro extended lispro stability out over 100 hours in both of the LS5 (FIG. 7A) and LGPhE5 (FIG. 7E) formulations. FIG. 7F provides a comparison of stability by aggregation times (t_{A}), defined as the time to a change in transmittance (λ=540 nm) of 10% or greater following stressed aging (i.e., continuous agitation at 37° C.). Data shown are average transmittance traces for n=3 samples per group and error bars are standard deviation.

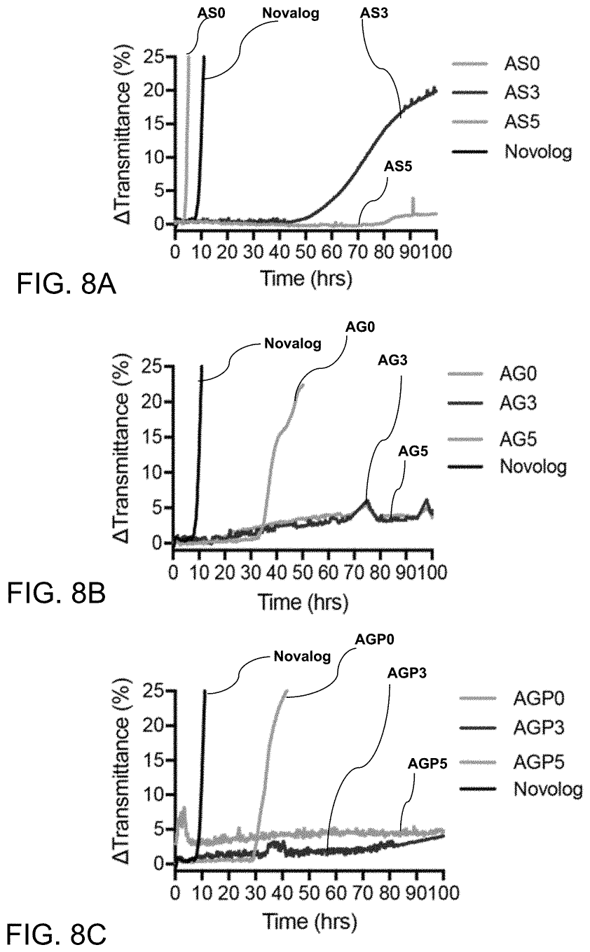

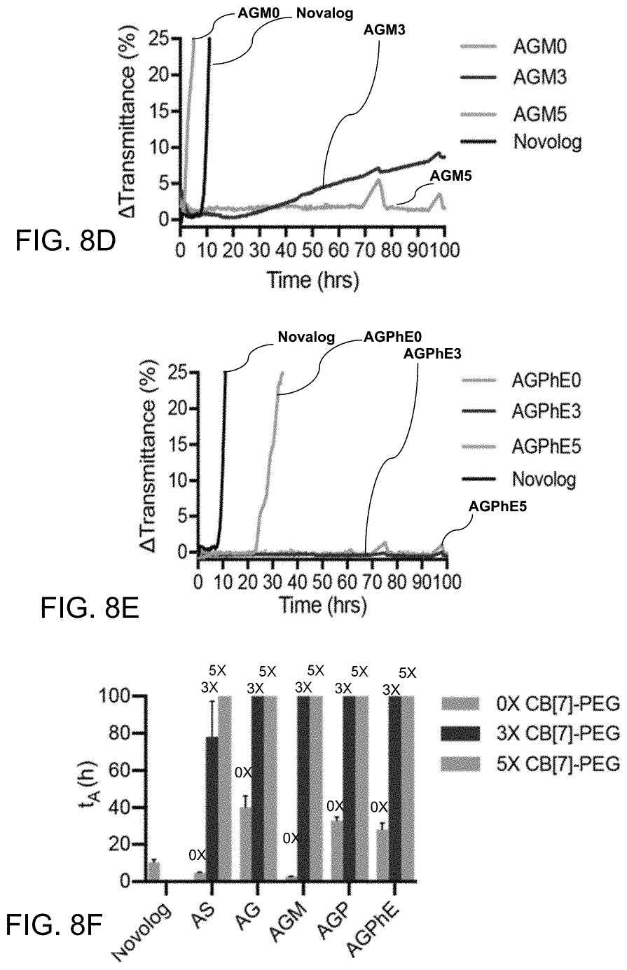

Zinc-free aspart was formulated under the same conditions as the zinc-free lispro: (i) saline (AS), (ii) glycerol (AG), (iii) glycerol/phenol (AGP), (iv) glycerol/meta-cresol (AGM), or (v) glycerol/phenoxyethanol (AGPhE) to evaluate stability. The results are plotted in FIGS. 8A-8E, respectively. Zinc-free aspart formulations were overall more stable than lispro formulations and the AG0, AGP0 and AGPhE0 formulations (FIGS. 8B, 8C and 8E) were more stable than the commercial Novolog formulation without the addition of CB[7]-PEG. A three-fold excess of CB[7]-PEG stabilized aspart over 100 hours in all formulation conditions except AS3. All formulations were stable over 100 hours with the five-fold excess of CB[7]-PEG. FIG. 8F provides a comparison of stability by aggregation times (t_{A}), defined as the time to a change in transmittance (λ=540 nm) of 10% or greater following stressed aging (i.e., continuous agitation at 37° C.). Data shown are average transmittance traces for n=3 samples per group and error bars are standard deviation. No difference in blood glucose depletion was observed in diabetic rats when animals were dosed with (i) Novolog or (ii) Zn-free aspart formulated with CB[7]-PEG₅ₖ (5 eq. with respect to insulin), indicating that formulation of monomeric insulin with CB[7]-PEG does not alter bioactivity or pharmacodynamics in vivo. FIG. 9 provides blood glucose curves after injection of commercial Novolog (black circle) or Zn-free aspart formulated with CB[7]-PEG (5 eq.) (grey square) in fasted diabetic rats (n=5-6). As indicated by the near overlap of the curves, the addition of CB[7]-PEG to Zn-free aspart formulations does not alter the duration of insulin action and maintains aspart bioactivity compared to commercial Novolog. Formulations were diluted 10× in phosphate buffered saline prior to injection to adjust doses.

### Example 3: Insulin Monomer Diffusion by DOSY

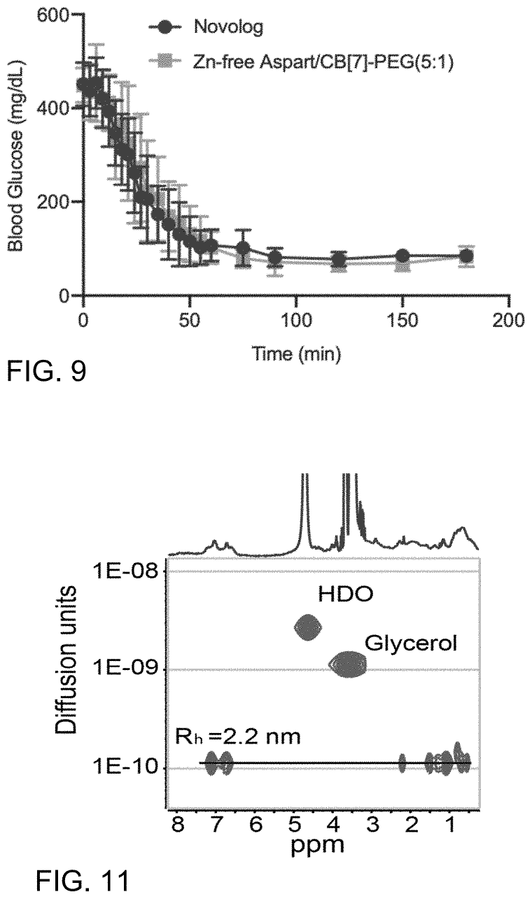

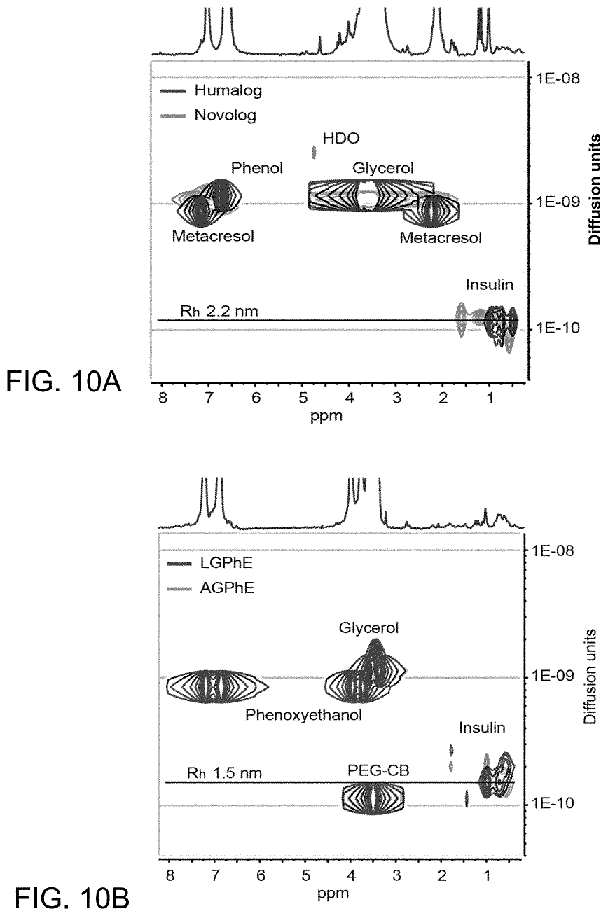

Diffusion-ordered NMR spectroscopy (DOSY) was used to provide insight into the size and diffusion characteristics of insulin lispro and aspart under LGPhE and AGPhE formulation conditions in the presence of CB[7]-PEG. Formulations comprising CB[7]-PEG could not be assessed with SEC-MALS on account of confounding alterations to the retention time of the insulin species on the chromatography column and in the light scattering. Formulating monomeric insulin analogues with CB[7]-PEG is expected to negligibly increase the diffusivity and corresponding hydrodynamic radius of the insulin in formulation compared to the insulin monomer alone on account of the highly dynamic nature of the CB[7]-insulin binding interaction. The diffusivities of the insulin molecules in zinc-free formulations of either LGPhE or AGPhE were compared against commercial Humalog and Novolog formulations. Humalog and Novolog both exhibited insulin diffusivities of 1.13×10⁻¹⁰ m²/s, corresponding to hydrodynamic radii of 2.2 nm (FIG. 10A), which is consistent with reported literature values. By using EDTA to remove zinc from commercial Novolog, the hydrodynamic radius remains unchanged at 2.2 nm, demonstrating that the absence of zinc alone it not sufficient to alter the insulin association state (FIG. 11). In contrast, the insulin molecules in zinc-free LGPhE and AGPhE formulations comprising CB[7]-PEG demonstrated increased diffusivities of 1.60×10⁻¹⁰ m²/s, corresponding to a hydrodynamic radius of 1.5 nm, which is significantly smaller than commercial insulin analogue formulations and approximately the same size as the reported literature value for the insulin monomer. (DOSY) provides insight into the formation of protein/CB[7]-PEG complexes and their rates of diffusion in formulation. Diffusion characteristics demonstrate that lispro and aspart diffuse at a similar rate under both commercial Humalog and Novolog in the presence of zinc ion (FIG. 10A) and LGPhE and AGPhE in the presence of CB[7]-PEG (0.6 eq) (FIG. 10B). Increased diffusion was observed for insulin LGPhE (FIG. 10C) and AGPhE (FIG. 10D) formulated with 0.6 eq. CB[7]-PEG compared to commercial formulation conditions. These observations suggest that while CB[7]-PEG stabilizes the monomeric form of insulin in formulation, the highly dynamic binding of CB[7]-PEG to insulin negligibly alters the diffusivity of the insulin molecule.

Excipient choice in insulin formulations is critical in determining insulin association state, stability, and the rate of absorption in vivo. At present, there is a need for insulin formulations that more closely mimic endogenous secretion, which ultimately requires insulin formulations that are more mono-disperse and primarily contain insulin monomers. In order to engineer the next generation of monomeric insulin formulations, an understanding of the effect of excipient choices on insulin association state is necessary. In this study, commonly used parenteral preservatives were evaluated for their propensity to increase the ratio of insulin monomers in formulation, and with the assistance of a stabilizing excipient, CB[7]-PEG, enhance insulin stability. We have identified that a formulation containing glycerol as a tonicity agent and phenoxyethanol as a preservative is the optimal combination to promote the insulin monomer. whereby upwards of 85% of the insulin is in a monomer state. This formulation exhibits over 10-fold extended stability compared to commercial formulations when formulated with CB[7]-PEG. Moreover, DOSY NMR highlights that CB[7]-PEG binding to insulin does not significantly impact the diffusivity of insulin or its association state. The increased insulin monomer composition in these formulations can potentially enable ultra-fast onset of insulin action combined with short duration of action to allow for meal-time responsiveness with reduced post-prandial hypoglycemic events.

### Example 4: CB[7]-PEG Preparation

CB[7]-PEG was prepared according to published protocols, with method modification to enable copper “click” chemistry following reported protocols. (See, e.g., L. Zou, et al., “Dynamic Supramolecular Hydrogels Spanning an Unprecedented Range of Host—Guest Affinity”, ACS Appl. Mater. Inter. 2019, 11, 5695-5700.) Novolog (Novo Nordisk) and Humalog (Eli Lilly) were purchased and used as received. Zinc-free lispro and zinc-free aspart were isolated using PD MidiTrap G-10 gravity columns (GE Healthcare) and then concentrated using Amino Ultra 3K centrifugal units (Millipore). All other reagents were purchased from Sigma-Aldrich unless otherwise specified.

### Example 5: SEC-MALS

Number-averaged molecular weight (MW) and dispersity (Ð=Mw/Mn) of formulations were obtained using size exclusion chromatography (SEC) carried out using a Dionex Ultimate 3000 instrument (including pump, autosampler, and column compartment) outfitted with a Dawn Heleos II Multi Angle Ligh Scattering detector, and a Optilab rEX refractive index detector. The column was a Superose 6 Increase 10/300 GL from GE healthcare. Data was analyzed using Astra 6.0 software.

Zinc-free insulin lispro and aspart were evaluated under the following buffer conditions: (i) sodium chloride (0.9%), (ii) glycerol (2.6%), (iii) glycerol (2.6%) & meta-cresol (0.315%), and (iv) glycerol (2.6%) & phenoxyethanol (0.85%). Controls consisted of either (v) commercial Humalog formulation comprising glycerol (1.6%), meta-cresol (0.315%), dibasic sodium phosphate (0.188%), and zinc (0.00197%), or (vi) commercial Novolog formulation comprising glycerol (1.6%), meta-cresol (0.172%), phenol (0.15%), sodium chloride (0.058%), dibasic sodium phosphate (0.125%), and zinc (0.00196%). Insulin lispro and aspart were injected at a concentration of a minimum 36 mg/mL protein and a volume of 100 μL. A do/dc of 0.186 mL/g was used for all samples. The resulted in max peak concentrations ranging from 3.0 mg/mL to 4.3 mg/mL, depending of protein oligomerization equilibria.

The molar fraction of monomeric, dimeric and hexameric insulin was determined by fitting experimentally derived number-averaged (Mn) and weight-averaged (Mw) molecular weights determined by SEC-MALS to Equation 1 and Equation 2, where m, d and h, respectively, represent the molar fractions of monomeric, dimeric and hexameric insulin while I represents the molecular weight of monomeric insulin (5831 g/mol). The solver was constrained so that m+d+h=1 while m, d and h remain between 0 and 1.

$${M}_{n} = m * I + d * 2 I + h * 6 I \qquad ( 1 )$$

$${M}_{w} = \frac{( m * {I}^{2} + d * 4 {I}^{2} + h * 36 {I}^{2} )}{m * I + d * 2 I + h * 6 I} \qquad ( 2 )$$

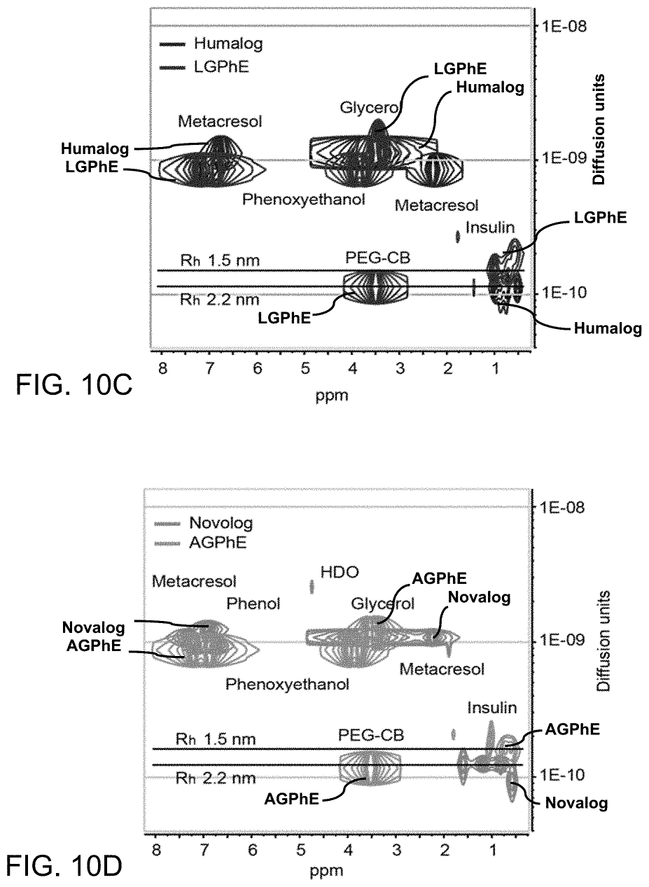

FIGS. 12A and 12B respectively plot the weight-averaged molecular weight of the zinc-free insulin lispro and aspart association states when formulated in (i) phosphate buffer, sodium chloride (0.9%) (LS, AS), (ii) phosphate buffer with glycerol (2.6%) (LG, AG), (iii) phosphate buffer with glycerol (2.6%) and meta-cresol (0.315%) (LGM, AGM), and (iv) phosphate buffer with glycerol (2.6%) and phenoxyethanol (0.85%) (LGPhE, AGPhE). Formulations were compared against a formulation of commercial Humalog or Novolog.

### Example 6: In Vitro Stability

Methods for aggregation assays for recombinant human insulin were adapted from Webber et al. (P. Nat. Acad. Sci. U.S.A 2016, 113, 14189). Briefly, formulation samples were plated at 150 μL per well (n=3/group) in a clear 96-well plate and sealed with optically clear and thermally stable seal (VWR). The plate was immediately placed into a plate reader and incubated with continuous shaking 37° C. Absorbance readings were taken every 10 minutes at 540 nm for 100 h (BioTek SynergyH1 microplate reader). The aggregation of insulin leads to light scattering, which results in reduction of sample transmittance. The time for aggregation was defined as a>10% increase in transmittance from the transmittance at time zero. Zinc(II) was removed from the insulin lispro and insulin aspart through competitive binding by addition of ethylenediaminetetraacetic acid (EDTA), which exhibits a dissociation binding constant approaching attomolar concentrations (K_{D}˜10⁻¹⁸ M).^{[41, 42]} EDTA was added to formulations (4 eq with respect to zinc) to sequester zinc from the formulation and then removed using a PD-10 desalting column (GE Healthcare) and then concentrated using Amino Ultra 3K centrifugal units (Millipore). Insulin lispro or aspart concentration was then measured by ELISA (Mercodia, Iso-Insulin ELISA) and then formulation excipients were added. All lispro or aspart formulations were formulated in phosphate buffer (pH=7.4) with the following combinations of excipients i) sodium chloride (0.9%), ii) glycerol (2.6%), iii) glycerol (2.6%) & phenol (0.25%), iv) glycerol (2.6%) & meta-cresol (0.315%), v) glycerol (2.6%) & phenoxyethanol (0.85%). CB[7]-PEG was added to formulations at either three or five molar equivalents with respect to insulin. Controls included commercial formulations of Novolog (insulin aspart) and Humalog (insulin lispro).

### Example 7: NMR DOSY

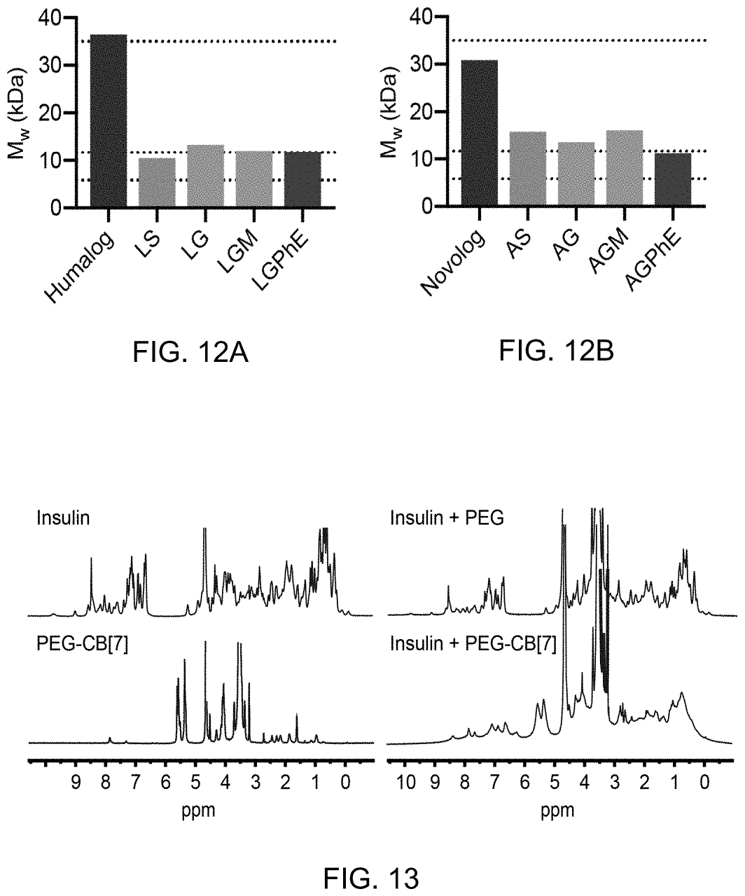

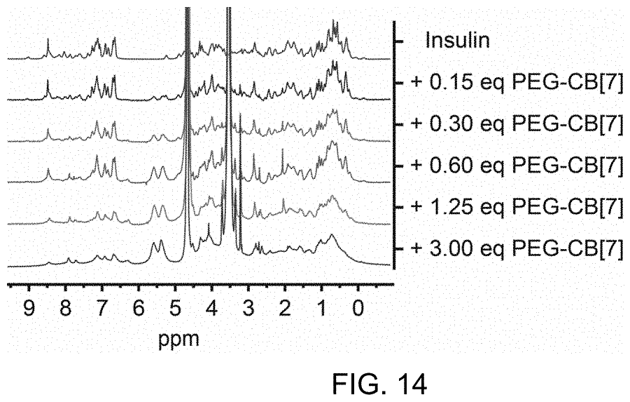

¹H 2D DOSY spectra were recorded at a protein concentration (lispro or aspart) of 3.4 mg/mL with 50-60% D₂O under the following conditions: (i) commercial Novolog [glycerol (1.6%), meta-cresol (0.175%), phenol (0.15%), sodium chloride (0.058%), dibasic sodium phosphate (0.125%), zinc (0.00196%)], (ii) commercial Humalog [glycerol (1.6%), meta-cresol (0.315%), dibasic sodium phosphate (0.188%), and zinc (0.00196%)], (iii) lispro/glycerol/phenoxyethanol (LGPhE) [phosphate buffer, glycerol (2.6%), and phenoxyethanol (0.85%)], and (iv) aspart/glycerol/phenoxyethanol (AGPhE) [phosphate buffer, glycerol (2.6%), and phenoxyethanol (0.85%)]. 1D ¹H-NMR of the insulin/CB[7]-PEG complex showed a broadening of both insulin and CB[7]-PEG signals (FIG. 13). This was exacerbated with an increasing ratio of CB[7]-PEG to insulin, as shown in FIG. 14. Insulin/CB[7]-PEG complex can be tracked by the emergence of the characteristic peak˜6.4 ppm. Broadening of all signals was observed in the complex, likely due to short T2 relaxation caused by quick exchange of CB[7]-PEG.

As such, an optimum ratio of CB[7]-PEG to insulin for DOSY was established to 0.60 mol. A Varian Inova 600 MHz NMR instrument was used to acquire the data. Magnetic field strengths ranging from 2 to 57 G cm⁻¹. The DOSY time and gradient pulse were set at 132 ms (Δ) and 3 ms (δ) respectively. All NMR data were processed using MestReNova 11.0.4 software.

### Example 8: Statistical Analysis

Insulin stability experiments were conducted with n=3 and the data are shown as the mean transmittance over time. The extracted time to aggregation (t_{A}) are plotted as mean±standard deviation. Rat studies were performed with n=5-6 rats for each treatment group and blood glucose results are reported as mean blood glucose±standard deviation. In vitro AKT activity assays were performed with n=3 and data are shown as mean±standard deviation of the relative pAKT normalized by the total AKT ([pAKT]/[AKT]). An EC₅₀ regression (log(agonist) vs. response (three parameters)) was plotted and an extra sum of squares F-test (alpha=0.05) was performed using GraphPad Prism 8.

### Example 9: Acridine Orange Binding Affinity

Unmodified CB[7] was purchased from Strem Chemicals and Acridine Orange (AO) was purchased from Sigma-Aldrich. Binding of CB[7] to aspart was assessed using the AO dye displacement assay, as previously described. Briefly, 6 μM of CB[7] and 8 μM AO were combined with 100 μL of aspart samples. Aspart samples were diluted to concentrations of 0, 0.01, 0.1, 0.3, 0.5, 1, 1.5, 2, 3, 4 μm in H₂O. Samples were incubated overnight in light-free conditions, and fluorescent spectra were collected on an BioTek SynergyH1 microplate reader, exciting at 485 nm and collecting the resulting fluorescent spectra from 495 to 650 nm. The decay in the peak of AO fluorescent signal was fit to a one-site competitive binding model (GraphPad Prism, version 6.0), using the CB[7]·AO equilibrium constant reported previously (K_{eq}=2×10⁵ M⁻¹), to determine binding constants of unmodified CB[7] to insulin.

### Example 10: In Vitro Insulin Cellular Activity Assay

C2C12 mouse muscle myoblasts (ATCC CRL-1772) were cultured to confirm insulin functional activity via the AKT phosphorylation pathway using AlphaLISA SureFire Ultra(Perkin-Elmer) kits for detection of phosphorylated AKT 1/2/3 (pS473) compared to total Akt1. Cells were confirmed to be free of mycoplasma contamination prior to use. Dulbecco's Modified Eagle's Medium (DMEM) with 4.5 g/L D-glucose, L-glutamine, and 110 mg/L sodium pyruvate (Gibco) was supplemented with 10% fetal bovine serum (FBS) and 5% penicillin-streptomycin to formulate complete culture media. Cells were seeded at a density of 25,000 cells/well in a volume of 200 μl/well in a 96-well tissue culture plate and grown for 24 hours. Prior to insulin stimulation, the cells were washed twice with 200 μl of unsupplemented DMEM and starved in 100 μl of unsupplemented DMEM overnight. The media was then removed and the cells were stimulated with 100 μl of insulin (i) Humalog, (ii) LisproPhE, (iii) Aged Humalog (4 days shaking at 37° C.), diluted in unsupplemented DMEM to the desired concentration, for 30 min while incubating at 37° C. Cells were washed twice with 100 μl of cold 1 X Tris-buffered saline before adding 100 μl of lysis buffer to each well and shaking for at least 10 minutes at room temperature to fully lyse cells. 30 μl of lysate was transferred to a 96-well white half-area plate for each assay. Assays were completed according to the manufacturer's protocol. Plates were incubated at room temperature and read 18-20 hours after the addition of the final assay reagents using a Tecan Infinite M1000 PRO plate reader. Results were plotted as a ratio of [pAKT]/[AKT] for each sample (n=3 cellular replicates) and an EC50 regression (log(agonist) vs. response (three parameters)) was plotted using GraphPad Prism 8.

### Example 11: Streptozotocin Induced Model of Diabetes in Rats

Male Sprague Dawley rats (Charles River) were used for experiments. Animal studies were performed in accordance with the guidelines for the care and use of laboratory animals; all protocols were approved by the Stanford Institutional Animal Care and Use Committee. The protocol used for STZ induction adapted from the protocol by Kenneth K. Wu and Youming Huan. Briefly, male Sprague Dawley rats 160-230 g (8-10 weeks) were weighed and fasted 6-8 hours prior to treatment with STZ. STZ was diluted to 10 mg/mL in the sodium citrate buffer immediately before injection. STZ solution was injected intraperitoneally at 65 mg/kg into each rat. Rats were provided with water containing 10% sucrose for 24 hours after injection with STZ. Rat blood glucose levels were tested for hyperglycemia daily after the STZ treatment via a tail vein blood collection using a handheld Bayer Contour Next glucose monitor (Bayer). Diabetes was defined as having 3 consecutive blood glucose measurements>400 mg/dL in non-fasted rats.

### Example 12: In Vivo Pharmacodynamics in Diabetic Rats

Diabetic rats were fasted for 6-8 hours. Rats were injected subcutaneously with either commercial Novolog or Zn-free aspart with CB[7]-PEG (5:1) at a dose of 1.5 U/kg. Insulins were diluted 10-fold in phosphate buffered saline before injection to allow for accurate dosing of small volumes. Before injection, baseline blood glucose was measured. Rats with a baseline blood glucose between 400 mg/dL-500 mg/dL were selected for the study. After injection, blood was sampled every 3 minutes for the first 30 minutes, then every 5 minutes for the next 30 minutes, then at 75, 90, 120, 150, and 180 minutes. Blood glucose was measured using a handheld blood glucose monitor (Bayer Contour Next).

The embodiments provided herein are not to be limited in scope by the specific embodiments provided in the examples which are intended as illustrations of a few aspects of the provided embodiments and any embodiments that are functionally equivalent are encompassed by the present disclosure. Indeed, various modifications of the embodiments provided herein are in addition to those shown and described herein will become apparent to those skilled in the art and are intended to fall within the scope of the appended claims.

### REFERENCES (INCORPORATED HEREIN BY REFERENCE)

[1] WHO, Diabetes: Key Facts, World Health Organization. 2017.
[2] R. Mo, T. Jiang, J. Di, W. Tai, Z. Gu, Chem. Soc. Rev. 2014, 43, 3595.
[3] T. Heise, E. Zijlstra, L. Nosek, T. Rikte, H. Haahr, Diabetes Obes. Metab. 2017, 19, 208.
[4] D. M. Maahs, N. A. West, J. M. Lawrence, E. J. Mayer-Davis, Endocrinol. Metab. Clin. North Am. 2010, 39, 481.
[5] P. Senior, I. Hramiak, Can. J. Diabetes. 2019, 43, 515.
[6] R. A. Heptulla, L. M. Rodriguez, L. Bomgaars, K. Mason, M. W. Haymond, Diabetes 2005, 54, A110.
[7] G. J. Ryan, L. J. Jobe, R. Martin, Clin. Ther. 2005, 27, 1500.
[8] L. L. Want, R. Ratner, Int. J. Clin. Pract. 2006, 60, 1522.
[9] M. C. Jones, Am. Fam. Physician 2007, 75, 1831.
[10] Z. Baghban Taraghdari, R. Imani, F. Mohabatpour, Macromol. Biosci. 2019, 19, e1800458.
[11] C. Mathieu, P. Gillard, K. Benhalima, Nat. Rev. Endocrinol. 2017, 13, 385.
[12] F. Holleman, J. B. L. Hoekstra, New Engl. J. Med. 1997, 337, 176.
[13] K. Gast, A. Schüler, M. Wolff, A. Thalhammer, H. Berchtold, N. Nagel, G. Lenherr, G. Hauck, R. Seckler, Pharm. Res. 2017, 34, 2270.
[14] Q. X. Hua, M. A. Weiss, J. Biol. Chem. 2004, 279, 21449.
[15] R. J. Woods, J. Alarcón, E. McVey, J. R. Pettis. J. Diabetes. Sci. Technol. 2012, 6, 265.
[16] Y. Xu, Y. Yan, D. Seeman, L. Sun, P. L. Dubin. Langmuir. 2012, 28, 579.
[17] M. A. Weiss, Vitam. Horm. 2009, 80, 33.
[18] P. D. Home, Diabetes Obes. Metab. 2012, 14, 780.
[19] T. J. Kamerzell, R. Esfandiary, S. B. Joshi, C. R. Middaugh, D. B. Volkin, Adv. Drug Deliver. Rev.2011, 63, 1118.
[20] B. M. Rayaprolu, J. J. Strawser, G. Anyarambhatla, Drug Dev. Ind. Pharm. 2018, 44, 1565.
[21] B. K. Meyer, A. Ni, B. Hu, L. Shi, I Pharm. Sci. 2007, 96, 3155.
[22] S. Saha, S. Deep, Phys. Chem. Chem. Phys. 2016, 18, 18934.
[23] U. Derewenda, Z. Derewenda, E. J. Dodson, G. G. Dodson, C. D. Reynolds, G. D. Smith, C. Sparks, D. Swenson, Nature 1989, 338, 594.
[24] M. C. Manning, D. K. Chou, B. M. Murphy, R. W. Payne, D. S. Katayama, Pharm. Res. 2010, 27, 544.
[25] S. Mitragotri, P. A. Burke, R. Langer, Nat. Rev. Drug Discov. 2014, 13, 655.
[26] C. Yang, D. Lu, Z. Liu, Biochemistry 2011, 50, 2585.
[27] L. H. Guerreiro, M. F. A. N. Guterres, B. Melo-Ferreira, L. C. S. Erthal, M. d. S. Rosa, D. Lourenco, P. Tinoco, L. M. T. R. Lima, AAPS PharmSciTech. 2013, 14, 1083.
[28] T. Sisnande, L. H. Guerreiro, R. R. Braga, L. Jotha-Mattos, L. C. S. Erthal, P. Tinoco, B. M. Ferreira, L. M. T. R. Lima, PLoS One 2015, 10, e0138803.
[29] F. M. Veronese, A. Mero, BioDrugs 2008, 22, 315.
[30] M. J. Webber, E. A. Appel, B. Vinciguerra, A. B. Cortinas, L. S. Thapa, S. Jhunjhunwala, L. Isaacs, R. Langer, D. G. Anderson, P. Nat. Acad. Sci. U.S.A 2016, 113, 14189.
[31] T. Hirotsu, T. Higashi, I. I. Abu Hashim, S. Misumi, K. Wada, K. Motoyama, H. Arima, Mol. Pharm. 2017, 14, 368.
[32] M. Bush, N. Bouley, A. R. Urbach, J. Am. Chem. Soc. 2005, 127, 14511.
[33] L. M. Heitmann, A. B. Taylor, P. J. Hart, A. R. Urbach, J. Am. Chem. Soc. 2006, 128, 12574.
[34] P. Rajgariah, A. R. Urbach, J. Incl. Phenom. Macro. 2008, 62, 251.

[35] J. J. Reczek, A. A. Kennedy, B. T. Halbert, A. R. Urbach, J. Am. Chem. Soc. 2009, 131, 2408.
[36] I. Jonassen, S. Havelund, T. Hoeg-Jensen, D. B. Steensgaard, P. O. Wahlund, U. Ribel, Pharm. Res. 2012, 29, 2104.
[37] A. K. Attri, C. Fernández, A. P. Minton, Biophys. Chem. 2010, 148, 23.
[38] A. K. Attri, C. Fernández, A. P. Minton, Biophys. Chem. 2010, 148, 28.
[39] M. H. Jensen, P.-O. Wahlund, J. K. Jacobsen, B. Vestergaard, M. van de Weert, S. Havelund, J. Chromatogr. B 2011, 879, 2945.
[40] D. B. Ludwig, J. N. Webb, C. Fernández, J. F. Carpenter, T. W. Randolph, Biotechnol. Bioeng. 2011, 108, 2359.
[41] G. Berthon, Handbook of metal-ligand interactions in biological fluids. Bioinorganic chemistry, Marcel Dekker, New York 1995.
[42] R. S. Waters, N. A. Bryden, K. Y. Patterson, C. Veillon, R. A. Anderson, Biol. Trace Elem. Res. 2001, 83, 207.
[43] J. L. Whittingham, S. Chaudhuri, E. J. Dodson, P. C. E. Moody, G. G. Dodson, Biochemistry 1995, 34, 15553.
[44] G. D. Smith, E. Ciszak, W. Pangborn, Protein Sci. 1996, 5, 1502.
[45] P. Banerjee, S. Mondal, B. Bagchi, J. Chem. Phys. 2019, 150, 084902.
[46] S. M. Patil, D. A. Keire, K. Chen, AAPS J. 2017, 19, 1760.
[47] G. G. Adams, A. Meal, P. S. Morgan, Q. E. Alzahrani, H. Zobel, R. Lithgo, M. S. Kok, D. T. M. Besong, S. I. Jiwani, S. Ballance, S. E. Harding, N. Chayen, R. B. Gillis, PLoS One 2018, 13, e0195010.
[48] L. Zou, A. S. Braegelman, M. J. Webber, ACS Appl. Mater. Inter. 2019, 11, 5695.
[49] M. J. Webber, E. A. Appel, B. Vinciguerra, A. B. Cortinas, L. S. Thapa, S. Jhunjhunwala, L. Isaacs, R. Langer, D. G. Anderson, P. Nat. Acad. Sci. U.S.A 2016, 113, 14189.
[50] J. M. Chinai, A. B. Taylor, L. M. Ryno, N. D. Hargreaves, C. A. Morris, P. J. Hart, A. R. Urbach, J. Am. Chem. Soc. 2011, 133, 8810.
[51] K. K. Wu, Y. Huan, Curr. Protoc. Pharmacol. 2008, 40, 5.47.1.

41. A pharmaceutical composition comprising:
a) insulin or an insulin analog;
b) an optionally substituted cucurbit[7]uril (CB[7])—polyethylene glycol (PEG) conjugate;
c) a tonicity agent; and
d) a preservative;
wherein at least one of:
no more than 20% of the insulin or insulin analogue exists as hexamers; and
the preservative is phenoxyethanol.

42. The pharmaceutical composition of claim 41, comprising an insulin analog selected from the group consisting of insulin aspart, insulin lispro, and insulin glulisine.

43. The pharmaceutical composition of claim 41, wherein the tonicity agent is one or a combination of glycerol, sodium chloride, and mannitol.

44. The pharmaceutical composition of claim 41, wherein CB7 is conjugated to PEG via a linker.

45. The pharmaceutical composition of claim 44, wherein the linker is of Formula (L-1): 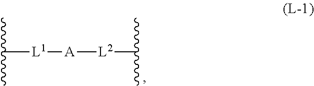
wherein:
each of L¹ and L² is independently a bond, optionally substituted alkylene, or optionally substituted heteroalkylene; and
A is a bond, an optionally substituted heterocyclyl, an optionally substituted heteroaryl, or an optionally substituted triazole moiety.

46. The pharmaceutical composition of claim 41, wherein the PEG has a molecular weight of less than about 1 kDa, from about 1 to about 10 kDa, from about 5 to about 10 kDa, from about 10 to about 30 kDa, more than about 30 kDa, or less than about 100 kDa.

47. The pharmaceutical composition of claim 41, further comprising at least one of:
a phosphate buffer, or
a sodium phosphate buffer.

48. The pharmaceutical composition of claim 41, wherein at least one of:
the pharmaceutical composition is substantially free of a stabilizing agent that promotes the formation or stability of insulin hexamer,
the pharmaceutical composition is substantially free of zinc, or the pharmaceutical composition is substantially free of polysorbate.

49. The pharmaceutical composition of claim 41, wherein at least one of:
the insulin or insulin analogue has a concentration of from about 50 U/mL to about 200 U/mL,
the insulin or insulin analogue has a concentration of about 100 U/mL,
the tonicity agent has an amount of from about 0.5% to about 5% of the total weight of the pharmaceutical composition, or
the tonicity agent has an amount of about 2.6% of the total weight of the pharmaceutical composition.

50. The pharmaceutical composition of claim 41, wherein at least one of:
the preservative has an amount of from about 0.2% to about 1.5% of the total weight of the pharmaceutical composition,
the preservative has an amount of about 0.85% of the total weight of the pharmaceutical composition, or
the preservative is phenoxyethanol.

51. The pharmaceutical composition of claim 41, wherein at least one of:
the molar ratio of CB[7]—PEG to the insulin or insulin analogue is from about 1:1 to about 10, or
the molar ratio of CB[7]—PEG to the insulin or insulin analogue is from about 3:1 to about 5:1.

52. The pharmaceutical composition of claim 41, wherein at least one of:
at least 50% of the insulin or insulin analogue exists as monomers in the pharmaceutical composition,
at least 60% of the insulin or insulin analogue exists as monomers in the pharmaceutical composition,
no more than 20% of the insulin or insulin analogue exists as hexamers in the pharmaceutical composition, or
no more than 10% of the insulin or insulin analogue exists as hexamers in the pharmaceutical composition.

53. The pharmaceutical composition of claim 41, further comprising at least one of:
amylin or an amylin analogue, or
amylin or an amylin analogue comprising pramlintide.

54. The pharmaceutical composition of claim 41, wherein at least one of:
the pharmaceutical composition retains a minimum of 80% activity after stressed aging at 37° C. for a minimum of 24 hours, or
the pharmaceutical composition is suitable for subcutaneous administration.

55. A method of managing or reducing blood glucose level in a subject in need thereof, comprising administering a therapeutically effective amount of the pharmaceutical composition of claim 41 to the subject.

56. The method of claim 55, wherein the subject has either type 1 diabetes or type 2 diabetes.

57. The method of claim 55, wherein the pharmaceutical composition is administered subcutaneously.

58. A kit, comprising:
the pharmaceutical composition of claim 41; and
an instruction for treating diabetes or managing or reducing blood glucose level.

59. The kit of claim 58, further comprising at least one of:
a pump configured to deliver the pharmaceutical composition to a subject;
a pen configured to deliver the pharmaceutical composition to a subject; or
a delivery device configured to deliver the pharmaceutical composition to a subject.
